(19) United States
(12) Patent Application Publication
(10) Pub. No.: US 2020/0143916 A1
(43) Pub. Date: May 7, 2020

(54) Devices, Systems, and Methods for Enhanced Patient Monitoring and Vigilance

(71) Applicant: Georgia Tech Research Corporation, Atlanta, GA (US)

(72) Inventors: Eva K. Lee, Atlanta, GA (US); Zhuonan Li, Atlanta, GA (US)

(21) Appl. No.: 16/676,348

(22) Filed: Nov. 6, 2019

Related U.S. Application Data
(60) Provisional application No. 62/756,545, filed on Nov. 6, 2018.

(51) Int. Cl. G16H 10/60; G16H 40/67

(52) U.S. Cl. CPC G16H 10/60; G16H 40/67

(57) ABSTRACT

A method including: receiving updates to patient vitality information from a plurality of health monitors; determining whether the patient vitality information has changed; and responsive to determining that the patient vitality information has changed, pushing, to a user device, a notification indicative of the changed vitality information

### CROSS-REFERENCE TO RELATED APPLICATIONS

This application claims priority to U.S. Provisional Patent Application Ser. No. 62/756,545, filed on Nov. 6, 2018, which is incorporated herein by reference in its entirety as if fully set forth below.

### STATEMENT REGARDING FEDERALLY SPONSORED RESEARCH OR DEVELOPMENT

This invention was made with government support under Award No. 1361532 awarded by the National Science Foundation (NSF). The government has certain rights in the invention.

### FIELD

This disclosure relates generally to patient monitoring and, more specifically, to devices, systems, and methods of remotely monitoring patient vitals with enhanced vigilance.

### BACKGROUND

In modern healthcare facilities, various health monitors are hooked to patients. The health monitors capture various vitality measures such as blood pressure (BP), heart rate (HR), and end-tidal CO₂ (EtCO2) and SpO₂, an estimate of arterial oxygen saturation. This information is useful for healthcare professionals to monitor the health of patients, and to quickly detect and respond to emergency situations. However, healthcare professionals cannot stay in a room with the patient at all time. For example, physician anesthesiologists provide continuous pain relief and sustain patients' critical life functions as the patients are affected throughout surgical, obstetrical, or other medical procedures. Physician anesthesiologists often head an Anesthesia Care Team including qualified nonphysician anesthesia providers and/or resident physicians in training in the provision of anesthesia care. Typically, the anesthesiologist makes rounds from one operating room to the next, checking on each surgical case frequently. In other words, the anesthesiologist is often absent from an operating room during a surgery. In the related art, if a patient experiences an emergency while the physician is absent, nurses in the operating room must locate the physician, explain the situation, and then have the physician return to the operating room to assess and handle the situation. This process relies heavily upon human effort and can create a long response time gap before actions could be taken.

Accordingly, despite the benefits of modern health monitors, serious drawbacks exist in their efficacy and utility. In the related art, alert systems leverage electronic medical records (EMR) to trigger send updates to physicians. However, such systems are overly complex (e.g., owing to the necessity of securing patient health information and avoiding unintentional corruption of EMR data) and slow (owing to the large amount of EMR data necessary to review). Moreover, such systems are not capable of modularity, but must be deeply embedded within each hospitals' EMR system.

Thus, there is an unmet need for new and improved devices, systems, and methods for enhanced patient monitoring and vigilance that are adaptable and scalable. Aspects of the present disclosure relate to these and other issues.

### SUMMARY

The disclosed technology provides systems, devices, and methods for patient monitoring. In an embodiment, there is provided a method including: receiving updates to patient vitality information from a plurality of health monitors; determining whether the patient vitality information has changed; and responsive to determining that the patient vitality information has changed, pushing, to a user device, a notification indicative of the changed vitality information.

According to an embodiment, there is provided A method including: receiving an indication of specific rooms or monitor sets; outputting, for transmission to a monitoring server, data indicative of the specific rooms or monitor sets; receiving, from the monitoring server, an indication of patient vitality data corresponding to the specific rooms or monitor sets; updating, based on the received indication of patient vitality data, an application display indicating current patient vitality data; comparing the received indication of patient vitality data to preset thresholds; and response to the received indication of patient vitality data indicating that the patient vitality data is outside the preset thresholds, outputting, for display on the application display, an alert indicating an emergency associated with one of the specific rooms or monitor sets.

According to an embodiment, there is provided a system including: a processor; a transceiver; and a memory having stored thereon instructions that, when executed by the processor, control the processor to: receive, via the transceiver and from a plurality of health monitors, updates to patient vitality information; determine whether the patient vitality information has changed; and responsive to determining that the patient vitality information has changed, push, via the transceiver and to a user device, a notification indicative of the changed vitality information.

### BRIEF DESCRIPTION OF THE FIGURES

Reference will now be made to the accompanying Figures, which are not necessarily drawn to scale, and wherein:

FIGS. 1 and 2 illustrates environments in which aspects of the present disclosure may be implemented.

FIG. 3 is a timing diagram illustrating aspects of the present disclosure.

FIGS. 4 and 5 are flowchart of methods according to embodiments of the present disclosure.

FIG. 6 is a timing diagram illustrating aspects of the present disclosure.

FIGS. 7 and 8 are screenshots of an example application and application interface according to aspects the present disclosure.

FIG. 9 is an example computer architecture through which aspects of the present disclosure may be implemented.

### DETAILED DESCRIPTION

Some implementations of the disclosed technology will be described more fully with reference to the accompanying drawings. This disclosed technology may, however, be embodied in many different forms and should not be construed as limited to the implementations set forth herein.

According to some aspects of the present disclosure, a monitoring system retrieves vitality information from patient health monitors. The vitality information can be bundled such that all monitors of a single patient/room are packaged together. The monitoring system selectively captures the vitality measures to avoid patient-identifiable information. This reduces overhead and limits security requirements. The monitoring system analyzes updates to the vitality measures and, upon detecting a change, pushes the change to an application executing on a user device. The application reviews the updates, refreshes a user interface, and alerts the user if the changes to the vitality measures violate preset thresholds.

Example implementations of the disclosed technology will now be described with reference to the accompanying figures.

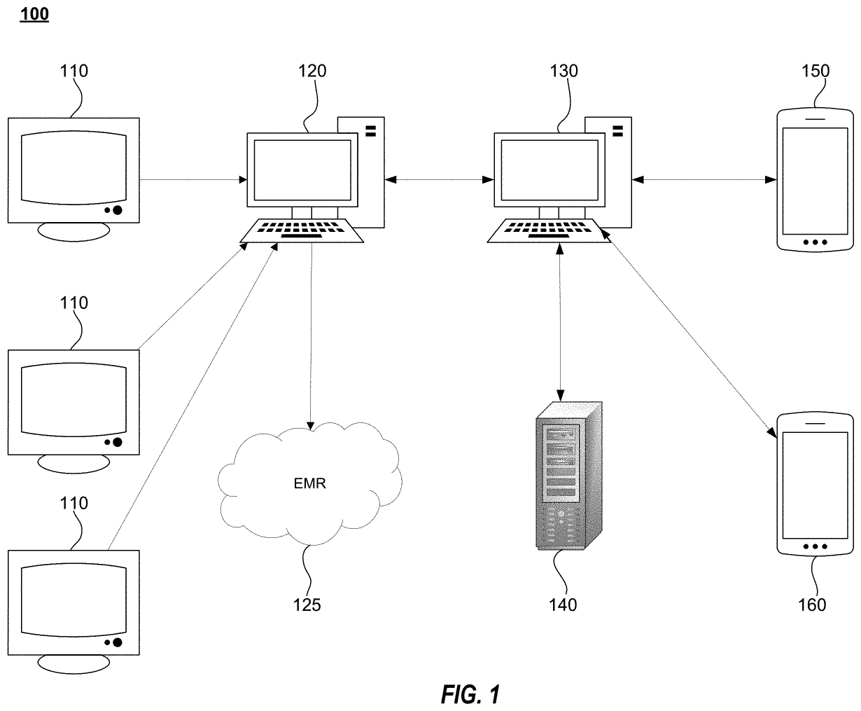

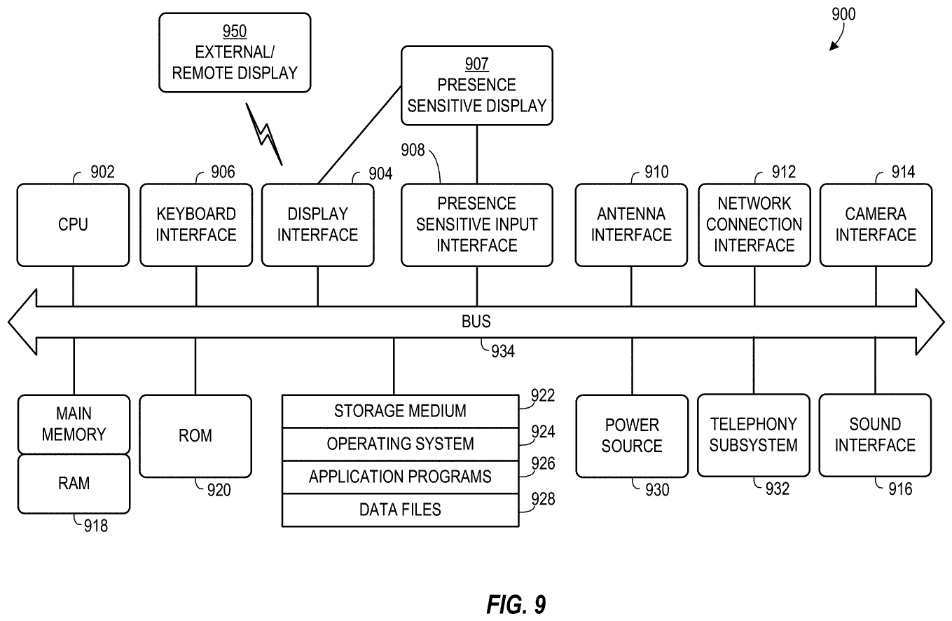

FIG. 1 illustrates a system environment 100 in which aspects of the invention can be implemented. Referring to FIG. 1, system environment 100 includes a plurality of patient monitors 110, a local collection system 120, an EMR system 125, a patient monitoring server 130 (e.g., monitoring server 130), a database 140, and user devices 150 and 160. In some examples, patient monitors 110, local collection system 120, EMR system 125, patient monitoring server 130, database 140, and/or user devices 150 and 160 can communicate with one another, e.g., through a physical or wireless connection or through data network and/or over an internet connection. Patient monitors 110, local collection system 120, EMR system 125, patient monitoring server 130, database 140, and/or user devices 150 and 160 can each include one or more processors, memories, and/or transceivers. As non-limiting examples, the user devices 150 and 160 can be cell phones, smartphones, laptop computers, tablets, smartwatches, or other personal computing devices that include the ability to communicate on one or more different types of networks. EMR system 125, monitoring server 130, and/or database 140 can include one or more physical or logical devices (e.g., servers, cloud servers, access points, switches, etc.) or drives. An example architecture that can be used to implement aspects of patient monitors 110, local collection system 120, EMR system 125, patient monitoring server 130, database 140, and/or user devices 150 and 160 are described below with reference to FIG. 9.

Patient monitors 110 may include one or more monitors configured to measure vitality characteristics of a patient such as blood pressure (BP), heart rate (HR), and end-tidal CO₂ (EtCO2) and SpO₂, an estimate of arterial oxygen saturation. Such monitors are regularly employed in operating rooms and patient rooms to track real-time vitality characteristics of a patient. Patient monitors 110 may display these measurements on related displays. In some implementations, patient monitors 110 may provide the real-time vitality information to local collection system 120, e.g., through a hardwire or network connection. Local collection system 120 provides this information to an EMR system 125, which links such information to a patient's electronic medical records. Furthermore, local collection system 120 can provide the health information to patient monitoring server 130.

Patient monitoring server 130 can retrieve the real-time vitality information from local collection system 120. In some cases, patient monitoring server 130 can access the data as it is transferred from local collection system 120 to EMR system 125. In an embodiment, patient monitoring server 130 can copy and/or extract a portion of the patient monitoring data collected by local collection system 120 without receiving and personal health information (i.e., without receiving any health information directly tied to a patient). Rather, as discussed below in greater detail, the vitality information may be associated with a particular room (e.g., operating room) and/or monitor suite. Accordingly, onerous EMR privacy rules and requirements can be avoided. The vitality information received by patient monitoring server 130 can utilize various formats, such as the Health Level 7 version 3 messaging standard. In an embodiment, patient monitoring server 130 updates database 140 as needed based on the real-time vitality information. Patient monitoring server 130 can update user devices 150 and 160 in accordance with updates to the vitality information. For example, if the blood pressure of a patient in an operating room drops, patient monitoring server 130 can push an update to an application executing on user device 150 to reflect the change.

As mentioned, user devices 150 and 160 can be embodied in various physical devices. For ease of explanation, user devices 150 and 160 can be thought of smartphones, though this is merely an example. User devices 150 and 160 may execute applications that are configured to communicate with monitoring server 130. The applications may enable a user to select a facility/location and specific rooms or monitoring suites associated with such facility. When registered with patient monitoring server 130, patient monitoring server 130 can push updates to the application for the selected rooms. When the updates hit pre-set thresholds (e.g., heart rate goes too high and/or erratic, or blood pressure drops too low), the application issues an alert on user devices 150 and 160. The user (e.g., doctor) can establish the preset thresholds through the application in a patient-specific manner. Furthermore, the application can alert the user to check on specific patient rooms and/or monitor suites. Furthermore, the color scheme of the application can be customized by the user. Therefore, the information displayed and alerts can mimic the displays of the various patient monitors 110, providing enhanced ease of use.

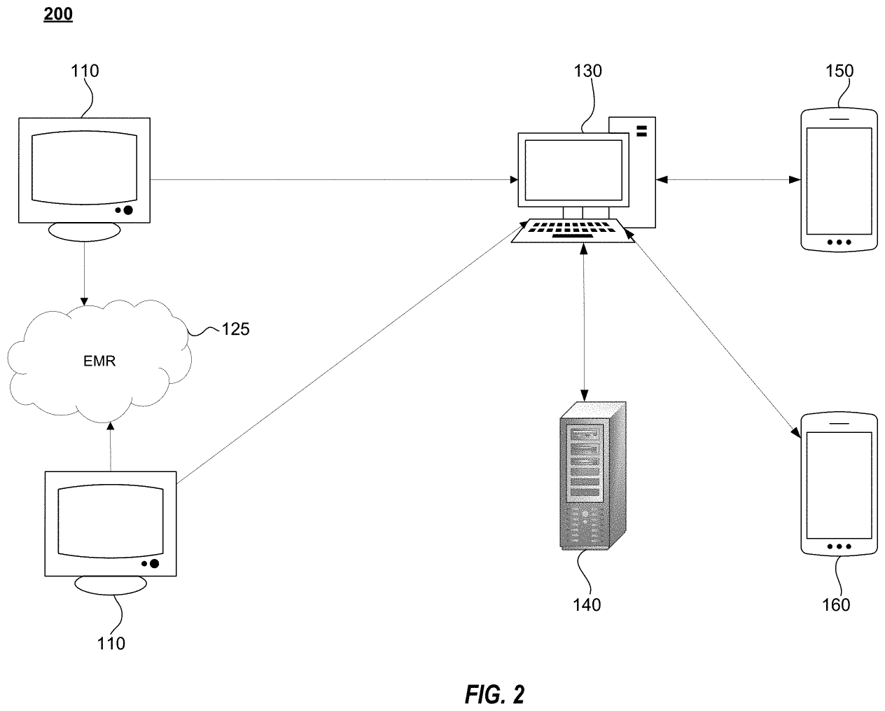

FIG. 2 illustrates a system environment 200 in which aspects of the invention can be implemented. Referring to FIG. 2, system environment 200 includes a plurality of patient monitors 110, an EMR system 125, a patient monitoring server 130 (e.g., monitoring server 130), a database 140, and user devices 150 and 160. Except as described below, patient monitors 110, EMR system 125, patient monitoring server 130 (e.g., monitoring server 130), database 140, and user devices 150 and 160 may be substantially similar in hardware and function as like elements described above with reference to FIG. 1. As will be noted, system environment 200 does not include local collection system 120. In such a case, patient monitors 110 provide the vitality information directly to EMR system 125. Likewise, monitoring server 130 retrieves the vitality data in substantial real-time from patient monitors 110. Such vitality data can be retrieved by direct wired and/or wireless connections, or through a network connection directly from monitors 110.

One of ordinary skill will recognize that environments 100 and 200 are merely examples. In some cases, various additional or alternative devices may be utilized, or functionality of several devices may be combined. For example, a system environment can further include a Wi-Fi adapter physically connected to a patient monitor 110, which transmits the vitality information to monitoring server 130.

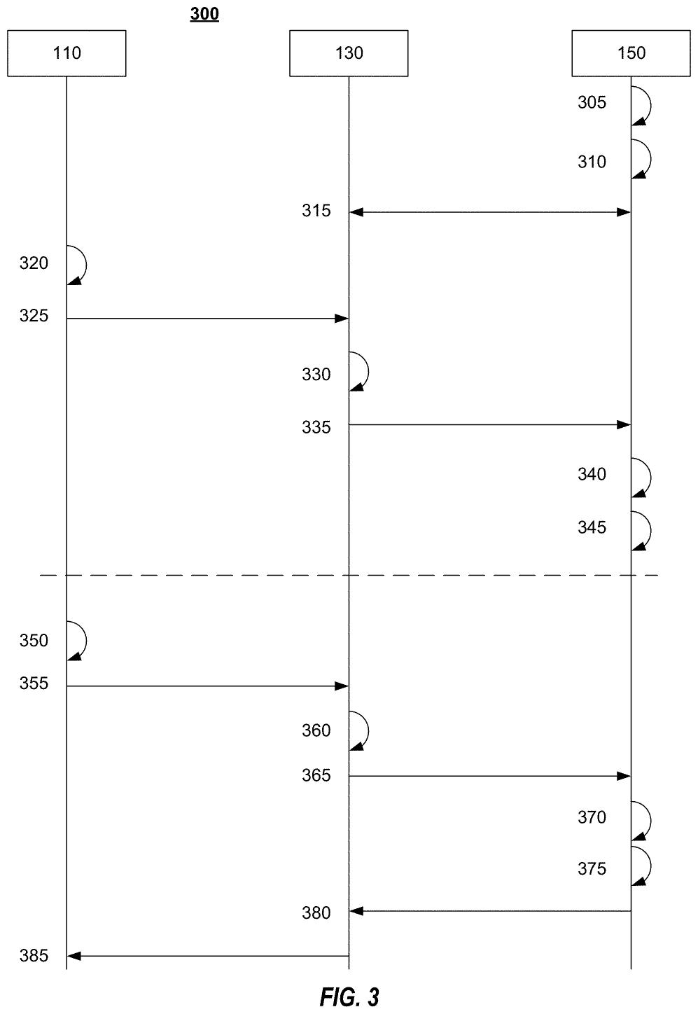

FIG. 3 is a timing diagram 300 of a method according to aspects of the present disclosure. In FIG. 3, certain components may be combined for simplicity of illustration. However, such functionality can be distributed in various ways to numerous systems. Referring to FIG. 3, user device 150 activates 305 a health monitoring application (e.g., in response to a user input), and receives 310 log-in credentials. User device 150 logs 315 into monitoring server 130. Logging into monitoring server 130 may include registering the health monitoring application executing on user device 150 to receive updates of patient vitality measures in specified rooms and/or associated with specific monitor suites.

Health monitor (or patent monitor) 110 measures 320 a patient's vitality and sends 325 the same to monitoring server 130. In some cases, monitoring server 130 may retrieve the vitality measurements from health monitor 110 and/or retrieve part of a stream provided to EMR system 125. In some cases, monitoring server 130 can selectively retrieve vitality measurements to avoid individualized patient health information, such as patient identifiers. Monitoring server 130 determines 330 whether an update to the vitality measure has occurred and, if so, pushes 335 the update to user device 150.

User device 150 refreshes 340 an interface of the health monitoring application. For example, health monitoring application may display vitality measurements associated with specific rooms. As the updates to the vitality measurements are received by user device 150, the application can update the interface to display the updated measurements. User device 150 can also determine that the updated measurement is outside of a pre-set threshold and alert 345 the user (e.g., outputting an alert on user device 150). Outputting the alert can include one or more of a pop-up message, a vibration, and/or an output sound. Once the alert is received, the user can go and check on the patient.

Health monitor 110 again measures 350 the patient's vitality and sends 355 the same to monitoring server 130. Monitoring server 130 determines 360 whether an update to the vitality measure has occurred and, if so, pushes 365 the update to user device 150. User device 150 refreshes 370 the interface of the health monitoring application with the updated information. User device 150 can also determine that the updated measurement is outside of a pre-set threshold and alert 375 the user (e.g., outputting an alert on user device 150). The user can determine that the alert is not needed (e.g., if the user is already in the room, or the problem has been resolved) and instruct 380 monitoring server 130 to stop the alert. In an embodiment, monitoring server 130 can likewise instruct 385 patient monitor 110 that the alert is not required. In some cases, patient monitor 110 may then update EMR system 125 with the response from the user.

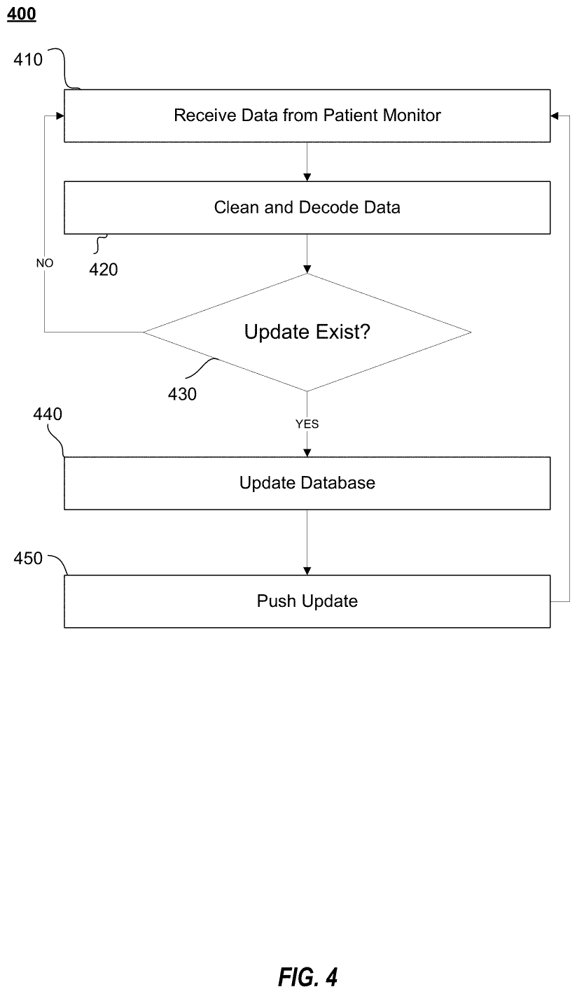

FIG. 4 is a flowchart 400 of an update method according to an example embodiment. The method described with reference to FIG. 4 may be implemented by monitoring server 130, though this merely an example. Monitoring server 130 receives 410 patient vitality data from patient monitor 110. In an embodiment, monitoring server 130 captures a data stream from patient monitor 110 to EMR system 125. In some cases, local collection system 120 may receive the patient vitality information from patient monitor 110 and transfer the vitality data to monitoring server 130. The data may be restricted to include only the vitality information (i.e., no patient-identifiable information) in order to avoid security requirements regarding electronic medical records.

Monitoring server 130 cleans and decodes 420 the patient health data. As will be understood by one of ordinary skill, in some cases, the patient health data from patient monitor 110 may be specially encoded (e.g., HL7 format) and/or include superfluous information. Accordingly, monitoring server 130 may be configured to decode the patient health data, clean the data of extraneous information, and format the same for storage in database 140 and/or use by an application on user device 150. Monitoring server 130 then compares the patient health data to previous reports to determine 430 if an update exists. If an update does not exist (430—no, patient monitoring server 130 receives updates on the patient vitality data. If the vitality information has been updated (430—yes), patient monitoring server 130 updates 440 database 140 with the information and pushes 450 the update to user device 150 and/or 160.

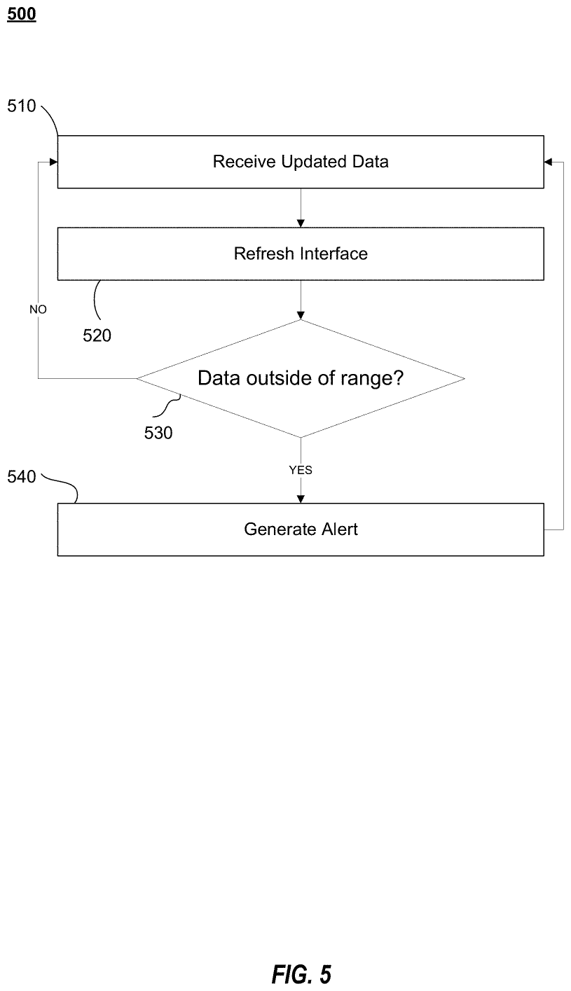

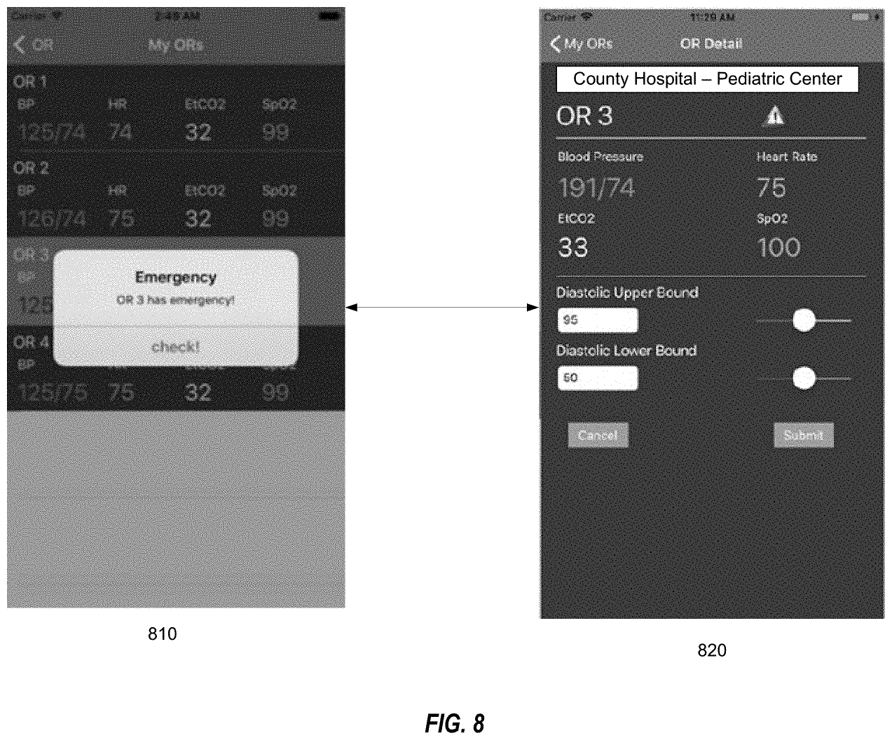

FIG. 5 is a flowchart 500 of an update method according to an example embodiment. The method described with reference to FIG. 5 may be implemented by user device 150/160, though this merely an example. User device 150 receives 510 updated patient vitality data from monitoring server 130. As a non-limiting example, monitoring server 130 may push updates to user device 150, such as through a push notification service. In response, user device 150 refreshes 520 an interface for a health monitoring application executing thereon. For example, as discussed below in greater detail with reference to FIG. 8, the application can display current vitality information for specific rooms and/or monitor suites. As the updates to the information is received the interface may display such updated information.

User device 150 determines 530 whether the updated patient health information is outside of a preset range. The preset range may be set by a user operating user device 150. For example, the preset ranges may include acceptable ranges of HR, BP, and body temperature detected by patient monitors 110. If the updated information is within the preset range (530—no), user device 150 may await a next update of patient vitality information. If the updated information is outside of the predetermined range (530—yes), user device 150 may generate 540 and output an alert to the user. Outputting the alert may include one or more of a pop-up message, highlighting on the user interface, generating and outputting an alert sound, and/or providing haptic feedback through the user device 150.

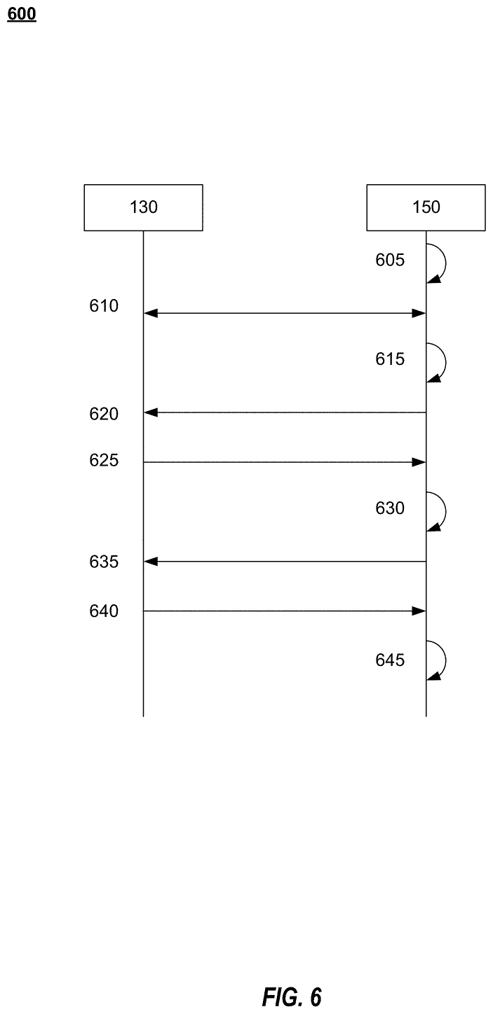

FIG. 6 is a timing diagram 600 of a method according to aspects of the present disclosure. In FIG. 6, certain components may be combined for simplicity of illustration. However, such functionality can be distributed in various ways to numerous systems. Referring to FIG. 6, user device 150 receives 605 user log-in credentials. User device 150 provides 610 the credentials to monitoring server 130, which validates the user and provides authorization to user device 150. A user (e.g., through a user interface) requests 615 a facility list/structure, and user device 150 requests 620 the same from monitoring server 130. Monitoring server 130 may retrieve a facility listing and/or blueprint from database 140, and provide 625 the blueprint to user device 150.

Based on the blueprint, user device 150 may display a facility structure. Based thereon, the user can select 630 specific rooms and/or monitor suites for notification. User device 150 provides 635 these selections to monitoring server 130. Monitoring server 130 then monitors the selected locations, and pushes 640 vitality updates to user device 150. Once received, user device 150 updates 645 an interface for the patient health application and executes any alerts determined to be necessary.

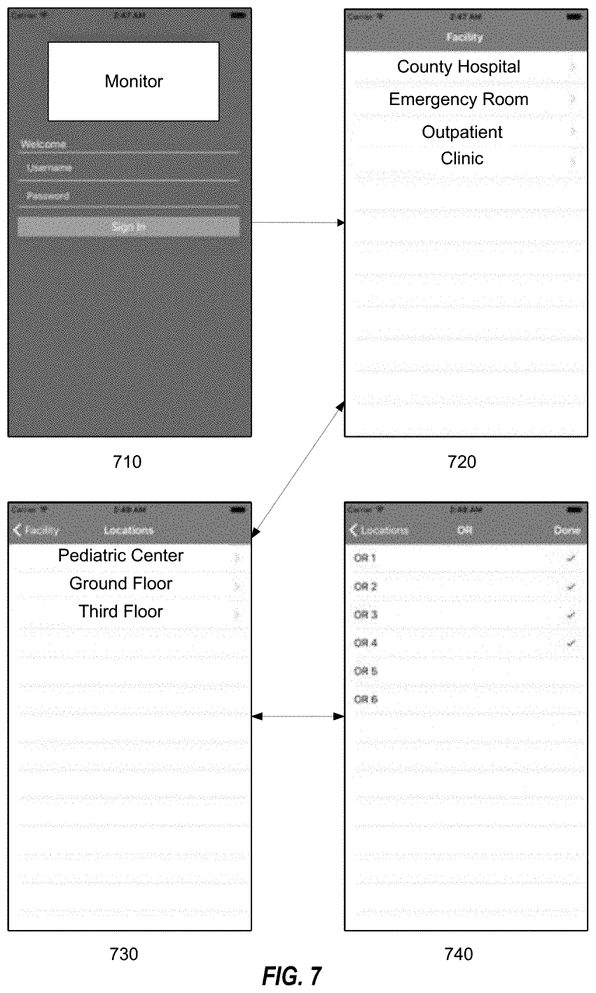

FIG. 7 are screenshots illustrating room selection of an example application and application interface according to aspects the present disclosure. The application may be executed, for example, on user device 150/160. In 710, the application presents a log-in page that can receive log-in credentials (e.g., username and password). At 720, the application presents a plurality of facility locations. A user can select one of the facility locations (e.g. county hospital) and will be presented with locations within the selected structure in 730 (e.g., pediatric center, ground floor, third floor). When the locations within the facility is selected by a user, the application displays a plurality of monitorable rooms and/or monitoring suites at 740. The user is then capable of selecting the rooms or other specific monitoring suites that the application should monitor.

FIG. 8 are screenshots illustrating alerts of an example application and application interface according to aspects the present disclosure. The application determines that an emergency exists in operating room 3 (OR3), and outputs an alert on user (810). The alert type (e.g., popup, sound, haptic feedback) and display format (e.g., color, font, location) may be customized by the user. When selected, application provides additional detail on the vitality measurements in OR3 (820). A user may select threshold ranges for various vitality measurements (e.g., an upper and lower bound for acceptable diastolic BP). Such ranges can be selected utilizing sliders, keyboard inputs, or other functions.

FIG. 9 is a block diagram of an illustrative computer system architecture 900, according to an example implementation. As non-limiting examples, portions of patient monitor 110, local collection system 120, EMR system 125, monitoring server 130, database 140, and user devices 150/160 may be implemented using one or more elements from the computer system architecture 900. It will be understood that the computing device architecture 900 is provided for example purposes only and does not limit the scope of the various implementations of the present disclosed systems, methods, and computer-readable mediums.

The computing device architecture 900 of FIG. 9 includes a central processing unit (CPU) 902, where computer instructions are processed, and a display interface 904 that acts as a communication interface and provides functions for rendering video, graphics, images, and texts on the display. In certain example implementations of the disclosed technology, the display interface 904 may be directly connected to a local display, such as a touch-screen display associated with a mobile computing device. In another example implementation, the display interface 904 may be configured for providing data, images, and other information for an external/remote display 950 that is not necessarily physically connected to the mobile computing device. For example, a desktop monitor may be used for mirroring graphics and other information that is presented on a mobile computing device. In certain example implementations, the display interface 904 may wirelessly communicate, for example, via a Wi-Fi channel or other available network connection interface 912 to the external/remote display 950.

In an example implementation, the network connection interface 912 may be configured as a communication interface and may provide functions for rendering video, graphics, images, text, other information, or any combination thereof on the display. In one example, a communication interface may include a serial port, a parallel port, a general-purpose input and output (GPIO) port, a game port, a universal serial bus (USB), a micro-USB port, a high definition multimedia (HDMI) port, a video port, an audio port, a Bluetooth port, a near-field communication (NFC) port, another like communication interface, or any combination thereof. In one example, the display interface 904 may be operatively coupled to a local display, such as a touch-screen display associated with a mobile device. In another example, the display interface 904 may be configured to provide video, graphics, images, text, other information, or any combination thereof for an external/remote display 950 that is not necessarily connected to the mobile computing device. In one example, a desktop monitor may be used for mirroring or extending graphical information that may be presented on a mobile device. In another example, the display interface 904 may wirelessly communicate, for example, via the network connection interface 912 such as a Wi-Fi transceiver to the external/remote display 950.

The computing device architecture 900 may include a keyboard interface 906 that provides a communication interface to a keyboard. In one example implementation, the computing device architecture 900 may include a presence-sensitive display interface 908 for connecting to a presence-sensitive display 907. According to certain example implementations of the disclosed technology, the presence-sensitive display interface 908 may provide a communication interface to various devices such as a pointing device, a touch screen, a depth camera, etc. which may or may not be associated with a display.

The computing device architecture 900 may be configured to use an input device via one or more of input/output interfaces (for example, the keyboard interface 906, the display interface 904, the presence-sensitive display interface 908, network connection interface 912, camera interface 914, sound interface 916, etc.) to allow a user to capture information into the computing device architecture 900. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 900 or may be a separate device. For example, the input device may be an accelerometer, a magnetometer, a digital camera, a microphone, and an optical sensor.

Example implementations of the computing device architecture 900 may include an antenna interface 910 that provides a communication interface to an antenna; a network connection interface 912 that provides a communication interface to a network. As mentioned above, the display interface 904 may be in communication with the network connection interface 912, for example, to provide information for display on a remote display that is not directly connected or attached to the system. In certain implementations, a camera interface 914 is provided that acts as a communication interface and provides functions for capturing digital images from a camera. In certain implementations, a sound interface 916 is provided as a communication interface for converting sound into electrical signals using a microphone and for converting electrical signals into sound using a speaker. According to example implementations, a random-access memory (RAM) 918 is provided, where computer instructions and data may be stored in a volatile memory device for processing by the CPU 902.

According to an example implementation, the computing device architecture 900 includes a read-only memory (ROM) 920 where invariant low-level system code or data for basic system functions such as basic input and output (I/O), startup, or reception of keystrokes from a keyboard are stored in a non-volatile memory device. According to an example implementation, the computing device architecture 900 includes a storage medium 922 or other suitable type of memory (e.g. such as RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 924, application programs 926 (including, for example, a web browser application, a widget or gadget engine, and or other applications, as necessary) and data files 928 are stored. According to an example implementation, the computing device architecture 900 includes a power source 930 that provides an appropriate alternating current (AC) or direct current (DC) to power components.

According to an example implementation, the computing device architecture 900 includes a telephony subsystem 932 that allows the device 900 to transmit and receive sound over a telephone network. The constituent devices and the CPU 902 communicate with each other over a bus 934.

According to an example implementation, the CPU 902 has appropriate structure to be a computer processor. In one arrangement, the CPU 902 may include more than one processing unit. The RAM 918 interfaces with the computer bus 934 to provide quick RAM storage to the CPU 902 during the execution of software programs such as the operating system application programs, and device drivers. More specifically, the CPU 902 loads computer-executable process steps from the storage medium 922 or other media into a field of the RAM 918 to execute software programs. Data may be stored in the RAM 918, where the data may be accessed by the computer CPU 902 during execution.

The storage medium 922 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 922, which may include a machine-readable storage medium.

According to one example implementation, the term computing device, as used herein, may be a CPU, or conceptualized as a CPU (for example, the CPU 902 of FIG. 9). In this example implementation, the computing device (CPU) may be coupled, connected, and/or in communication with one or more peripheral devices, such as display. In another example implementation, the term computing device, as used herein, may refer to a mobile computing device such as a Smartphone, tablet computer, or smart watch. In this example implementation, the computing device may output content to its local display and/or speaker(s). In another example implementation, the computing device may output content to an external display device (e.g., over Wi-Fi) such as a TV or an external computing system.

In example implementations of the disclosed technology, a computing device may include any number of hardware and/or software applications that are executed to facilitate any of the operations. In example implementations, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be used to receive or collect data and/or user instructions from a wide variety of input devices. Received data may be processed by one or more computer processors as desired in various implementations of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

Certain implementations of the disclosed technology are described above with reference to block and flow diagrams of systems and methods and/or computer program products according to example implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, implementations of the disclosed technology may provide for a computer program product, including a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology are described above with reference to mobile computing devices. Those skilled in the art recognize that there are several categories of mobile devices, generally known as portable computing devices that can run on batteries but are not usually classified as laptops. For example, mobile devices can include, but are not limited to portable computers, tablet PCs, Internet tablets, PDAs, ultra-mobile PCs (UMPCs) and smartphones.

In this description, numerous specific details have been set forth. It is to be understood, however, that implementations of the disclosed technology may be practiced without these specific details. In other instances, well-known methods, structures and techniques have not been shown in detail in order not to obscure an understanding of this description. References to “one implementation,” “an implementation,” “example implementation,” “various implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

An embodiment of the present disclosure may be implemented according to at least the following:

Clause 1: A method comprising: receiving updates to patient vitality information from a plurality of health monitors; determining whether the patient vitality information has changed; and responsive to determining that the patient vitality information has changed, pushing, to a user device, a notification indicative of the changed vitality information.

Clause 2: The method of Clause 1, wherein receiving the updates to the patient vitality information comprises extracting patient vitality information from a data stream provided to an electronic medical records (EMR) system.

Clause 3: The method of Clause 2, wherein extracting the patient vitality information comprises copying a portion of the data stream prior to receipt of the data stream by the EMR system.

Clause 4: The method of any of Clauses 1-3 further comprising: cleaning the received updates; and decoding the received updates.

Clause 5: The method of any of Clauses 1-4, wherein the updates to the patient vitality information do not include any patient-identifiable information.

Clause 6: The method of any of Clauses 1-5 further comprising: receiving, from the user device, an indication of specified rooms or monitor sets; and pushing the changed vitality information to the user device for the specified rooms or monitor sets.

Clause 7: The method of Clause 6 further comprising providing, to the user device, a facility listing, wherein receiving the indication of the specified rooms or monitor sets comprises receiving an indication of a selection of the specified rooms or monitor sets from within the facility listing.

Clause 8. The method of any of Clauses 1-7 further comprising, prior to pushing the changed vitality information to the user device, receiving, from the user device, log-in credentials, and validating the log-in credentials.

Clause 9: A method comprising: receiving an indication of specific rooms or monitor sets; outputting, for transmission to a monitoring server, data indicative of the specific rooms or monitor sets; receiving, from the monitoring server, an indication of patient vitality data corresponding to the specific rooms or monitor sets; updating, based on the received indication of patient vitality data, an application display indicating current patient vitality data; comparing the received indication of patient vitality data to preset thresholds; and response to the received indication of patient vitality data indicating that the patient vitality data is outside the preset thresholds, outputting, for display on the application display, an alert indicating an emergency associated with one of the specific rooms or monitor sets.

Clause 10: The method of Clause 9 further comprising: receiving, from the monitoring server, a facility listing; outputting, for display on the application display and based on the facility listing, identifiers of a plurality of medical facilities; receiving an indication of a selection of a facility of the plurality of medical facilities; outputting, for display on the application display, identifiers of a plurality of specific rooms or monitor sets corresponding to the selected medical facility; and receiving an indication of a selection of one or more of the displayed identifiers of the specific rooms or monitor sets, wherein outputting for transmission the data indicative of the specific rooms or monitor sets comprises outputting for transmission the indicative of the selected specific rooms or monitor sets.

Clause 11: The method of Clause 9 or 10 further comprising receiving an indication of thresholds for patient vitality data corresponding to a specified room or monitor set as the preset thresholds.

Clause 12: The method of any of Clauses 9-11 further comprising receiving an indication of alert and display color selections for at least one from among emergency notifications, vitality information, and urgent vitality information.

Clause 13: A system comprising: a processor; a transceiver; and a memory having stored thereon instructions that, when executed by the processor, control the processor to: receive, via the transceiver and from a plurality of health monitors, updates to patient vitality information; determine whether the patient vitality information has changed; and responsive to determining that the patient vitality information has changed, push, via the transceiver and to a user device, a notification indicative of the changed vitality information.

Clause 14: The system of Clause 13, wherein receiving the updates to the patient vitality information comprises extracting patient vitality information from a data stream provided to an electronic medical records (EMR) system.

Clause 15: The system of Clause 14, wherein extracting the patient vitality information comprises copying a portion of the data stream prior to receipt of the data stream by the EMR system.

Clause 16: The system of any of Clauses 13-15, wherein the instructions, when executed by the processor, further control the processor to: clean the received updates; and decode the received updates.

Clause 17: The system of any of Clauses 13-16, wherein the updates to the patient vitality information do not include any patient-identifiable information.

Clause 18: The system of any of Clauses 13-17, wherein the instructions, when executed by the processor, further control the processor to: receive, from the user device, an indication of specified rooms or monitor sets; and push the changed vitality information to the user device for the specified rooms or monitor sets.

Clause 19: The system of any of Clauses 13-18, wherein the instructions, when executed by the processor, further control the processor to provide, to the user device, a facility listing, wherein receiving the indication of the specified rooms or monitor sets comprises receiving an indication of a selection of the specified rooms or monitor sets from within the facility listing.

Clause 20: The system of any of Clauses 13-19, wherein the instructions, when executed by the processor, further control the processor to, prior to pushing the changed vitality information to the user device, receive, from the user device, log-in credentials, and validate the log-in credentials.

What is claimed is:

1. A method comprising:
receiving updates to patient vitality information from a plurality of health monitors;
determining whether the patient vitality information has changed; and
responsive to determining that the patient vitality information has changed, pushing, to a user device, a notification indicative of the changed vitality information.

2. The method of claim 1, wherein receiving the updates to the patient vitality information comprises extracting patient vitality information from a data stream provided to an electronic medical records (EMR) system.

3. The method of claim 2, wherein extracting the patient vitality information comprises copying a portion of the data stream prior to receipt of the data stream by the EMR system.

4. The method of claim 1 further comprising:
cleaning the received updates; and
decoding the received updates.

5. The method of claim 1, wherein the updates to the patient vitality information do not include any patient-identifiable information.

6. The method of claim 1 further comprising:
receiving, from the user device, an indication of specified rooms or monitor sets; and
pushing the changed vitality information to the user device for the specified rooms or monitor sets.

7. The method of claim 6 further comprising providing, to the user device, a facility listing, wherein receiving the indication of the specified rooms or monitor sets comprises receiving an indication of a selection of the specified rooms or monitor sets from within the facility listing.

8. The method of claim 1 further comprising, prior to pushing the changed vitality information to the user device, receiving, from the user device, log-in credentials, and validating the log-in credentials.

9. A method comprising:
receiving an indication of specific rooms or monitor sets;
outputting, for transmission to a monitoring server, data indicative of the specific rooms or monitor sets;
receiving, from the monitoring server, an indication of patient vitality data corresponding to the specific rooms or monitor sets;
updating, based on the received indication of patient vitality data, an application display indicating current patient vitality data;
comparing the received indication of patient vitality data to preset thresholds; and
response to the received indication of patient vitality data indicating that the patient vitality data is outside the preset thresholds, outputting, for display on the application display, an alert indicating an emergency associated with one of the specific rooms or monitor sets.

10. The method of claim 9 further comprising:
receiving, from the monitoring server, a facility listing;
outputting, for display on the application display and based on the facility listing, identifiers of a plurality of medical facilities;
receiving an indication of a selection of a facility of the plurality of medical facilities;
outputting, for display on the application display, identifiers of a plurality of specific rooms or monitor sets corresponding to the selected medical facility; and
receiving an indication of a selection of one or more of the displayed identifiers of the specific rooms or monitor sets, wherein outputting for transmission the data indicative of the specific rooms or monitor sets comprises outputting for transmission the indicative of the selected specific rooms or monitor sets.

11. The method of claim 9 further comprising receiving an indication of thresholds for patient vitality data corresponding to a specified room or monitor set as the preset thresholds.

12. The method of claim 9 further comprising receiving an indication of alert and display color selections for at least one from among emergency notifications, vitality information, and urgent vitality information.

13. A system comprising:
a processor;
a transceiver; and
a memory having stored thereon instructions that, when executed by the processor, control the processor to:
receive, via the transceiver and from a plurality of health monitors, updates to patient vitality information;
determine whether the patient vitality information has changed; and
responsive to determining that the patient vitality information has changed, push, via the transceiver and to a user device, a notification indicative of the changed vitality information.

14. The system of claim 13, wherein receiving the updates to the patient vitality information comprises extracting patient vitality information from a data stream provided to an electronic medical records (EMR) system.

15. The system of claim 14, wherein extracting the patient vitality information comprises copying a portion of the data stream prior to receipt of the data stream by the EMR system.

16. The system of claim 13, wherein the instructions, when executed by the processor, further control the processor to:
clean the received updates; and
decode the received updates.

17. The system of claim 13, wherein the updates to the patient vitality information do not include any patient-identifiable information.

18. The system of claim 13, wherein the instructions, when executed by the processor, further control the processor to:
receive, from the user device, an indication of specified rooms or monitor sets; and
push the changed vitality information to the user device for the specified rooms or monitor sets.

19. The system of claim 13, wherein the instructions, when executed by the processor, further control the processor to provide, to the user device, a facility listing, wherein receiving the indication of the specified rooms or monitor sets comprises receiving an indication of a selection of the specified rooms or monitor sets from within the facility listing.

20. The system of claim 13, wherein the instructions, when executed by the processor, further control the processor to, prior to pushing the changed vitality information to the user device, receive, from the user device, log-in credentials, and validate the log-in credentials.
